SEQUENCE LISTING (1 sequence)

SEQ ID NO: 1 - protein, 28 aa, Artificial sequence
  Thymosin alpha 1 peptide
SDAAVDTSSEITTKDLKEKKEVVEEAEN

(19) United States
(12) Patent Application Publication
(10) Pub. No.: US 2010/0311656 A1
(43) Pub. Date: Dec. 9, 2010

(54) TREATMENT OR PREVENTION OF RESPIRATORY VIRAL INFECTIONS WITH ALPHA THYMOSIN PEPTIDES

(75) Inventors: Alfred R. RUDOLPH, Los Altos Hills, CA (US); Cynthia W. TUTHILL, Menlo Park, CA (US)

Correspondence Address: COOLEY LLP;ATTN: Patent Group, Suite 1100, 777 - 6th Street, NW, WASHINGTON, DC, 20001, US

(73) Assignee: SciClone Pharmaceuticals, Inc., Foster City, CA (US)

(21) Appl. No.: 12/816,959

(22) Filed: Jun. 16, 2010

Related U.S. Application Data
(63) Continuation-in-part of application No. 10/553,317, filed on Sep. 26, 2006.
(60) Provisional application No. 60/464,645, filed on Apr. 23, 2003.
(60) Provisional application No. 60/470,420, filed on May 15, 2003.

(51) Int. Cl. A61K 38/22; A61P 31/14; A61P 37/04; A61P 31/12

(52) U.S. Cl. 514/12.9

(57) ABSTRACT

An alpha thymosin peptide is administered to a patient having, or at risk of a respiratory viral infection, coronavirus infection and/or SARS.

### CROSS-REFERENCE TO RELATED APPLICATIONS

This application is a continuation-in-part of U.S. application Ser. No. 10/553,317, filed Sep. 26, 2006, which is a National Phase of International Application Serial No. PCT/US2004/012663, filed Apr. 23, 2004, which claims the benefit of U.S. Provisional Application Ser. No. 60/464,645, filed Apr. 23, 2003 and U.S. application Ser. No. 60/470,420, filed May 15, 2003, all of which are hereby incorporated by reference in their entireties.

### DESCRIPTION OF THE TEXT FILE SUBMITTED ELECTRONICALLY

The contents of the text file submitted electronically herewith are incorporated herein by reference in their entirety: A computer readable format copy of the Sequence Listing (filename: SCIC-053_{—}02US_SeqList_ST25.txt, date recorded: Jun. 16, 2010, file size 1 kilobyte).

### FIELD OF THE INVENTION

The present invention relates to the field of treatment of respiratory viral infections.

### DESCRIPTION OF THE BACKGROUND ART

Severe acute respiratory syndrome (SARS) is a viral respiratory illness caused by a coronavirus, called SARS-associated corona virus (SARS-CoV). SARS was first reported in Asia in February 2003. Over the next few months, the illness spread to more than two dozen countries in North America, South America, Europe, and Asia.

In general, SARS begins with a high fever (temperature greater than 100.4° F. [>38.0° C.]). Other symptoms may include headache, an overall feeling of discomfort, and body aches. Some people also have mild respiratory symptoms at the outset. About 10 percent to 20 percent of patients have diarrhea. After 2 to 7 days, SARS patients may develop a dry cough. Most patients develop pneumonia.

The main way that SARS seems to spread is by close person-to-person contact. The virus that causes SARS is thought to be transmitted most readily by respiratory droplets (droplet spread) produced when an infected person coughs or sneezes. Droplet spread can happen when droplets from the cough or sneeze of an infected person are propelled a short distance (generally up to 3 feet) through the air and deposited on the mucous membranes of the mouth, nose, or eyes of persons who are nearby. The virus also can spread when a person touches a surface or object contaminated with infectious droplets and then touches his or her mouth, nose, or eye(s). In addition, it is possible that the SARS virus might spread more broadly through the air (airborne spread) or by other ways that are not now known.

According to the World Health Organization (WHO), a total of 8,098 people worldwide became sick with SARS during the 2003 outbreak. Of these, 774 died.

There remains a need in the art for the treatment or prevention of respiratory viral infections such as SARS.

### SUMMARY OF THE INVENTION

In accordance with the present invention, a method of treatment or prevention of a respiratory viral infection in a patient comprises administering to the patient an effective amount of an alpha thymosin peptide.

### DETAILED DESCRIPTION OF THE INVENTION

In accordance with one embodiment, the present invention relates to treatment or prevention of respiratory viral infections by administering an alpha thymosin peptide to a patient.

In accordance with another embodiment, the invention relates to treatment or prevention of coronavirus infection by administering an alpha thymosin peptide to a patient.

In accordance with a further embodiment, the invention relates to treatment or prevention of Severe Acute Respiratory Syndrome (SARS) in a patient by administering an alpha thymosin peptide.

Administration for prevention can be to persons at high risk because of contact with suspected disease carriers, or in carriers who are asymptomatic.

Alpha thymosin peptides comprise thymosin alpha 1 (TA1) peptides including naturally occurring TA1 as well as synthetic TA1 and recombinant TA1 having the amino acid sequence of naturally occurring TA1, amino acid sequences substantially similar thereto, or an abbreviated sequence form thereof, and their biologically active analogs having substituted, deleted, elongated, replaced, or otherwise modified sequences which possess bioactivity substantially similar to that of TA1, e.g., a TA1 derived peptide having sufficient amino acid homology with TA1 such that it functions in substantially the same way with substantially the same activity as TA1.

Administration can be by any suitable method, including injection, periodic infusion, continuous infusion, and the like. Suitable dosages of the alpha thymosin peptide can be in the range of about 0.001-10 mg/kg/day.

According to one aspect of this embodiment of the present invention, the dosage unit comprising an alpha thymosin peptide is administered to the patient on a routine basis. For example, the dosage unit can be administered more than once daily, once daily, weekly, monthly, etc. The dosage unit may be administered on a bi-weekly basis, i.e., twice a week, for example, on Tuesday and Saturday. The dosage unit of TA1 may be administered on a thrice weekly basis, i.e., three times per week.

Because the plasma half-life of subcutaneously injected TA1 is only about two hours, according to one embodiment, a TA1 peptide such as TA1 is administered to a patient in need of immune stimulation so as to substantially continuously maintain an immune stimulating-effective amount of the TA1 peptide in the patient's circulatory system during a substantially longer treatment or prevention period. Although much longer treatment periods are contemplated in accordance with the present invention, embodiments of the invention include substantially continuously maintaining an immune stimulating-effective amount of the TA1 peptide in the patient's circulatory system during treatment periods of at least about 6, 10, 12 hours, or longer. In other embodiments, treatment periods are for at least about a day, and even for a plurality of days, e.g., a week or longer. However, it is contemplated that treatments, as defined above, in which immune stimulating-effective amounts of the TA1 peptide are substantially continuously maintained in the patient's circulatory system, may be separated by non-treatment periods of similar or different durations.

In accordance with one embodiment, the TA1 peptide is continuously infused into a patient, e.g., by intravenous infusion, during the treatment period, so as to substantially continuously maintain an immune stimulating-effective amount of the TA1 peptide in the patient's circulatory system. The infusion may be carried out by any suitable means, such as by minipump.

Alternatively, an injection regimen of the TA1 peptide can be maintained so as to substantially continuously maintain an immune stimulating-effective amount of the TA1 peptide in the patient's circulatory system. Suitable injection regimens may include an injection every 1, 2, 4, 6, etc. hours, so as to substantially continuously maintain the immune stimulating-effective amount of the Thymosin alpha 1 peptide in the patient's circulatory system during the treatment period.

Although it is contemplated that during continuous infusion of the TA1 peptide, administration will be for a substantially longer duration, according to one embodiment the continuous infusion of the TA1 peptide is for a treatment period of at least about 1 hour. More preferably, continuous infusion is carried out for longer periods, such as for periods of at least about 6, 8, 10, 12 hours, or longer. In other embodiments, continuous infusion is for at least about one day, and even for a plurality of days such as for one week or more.

In preferred embodiments, the TA1 peptide is present in a pharmaceutically acceptable liquid carrier, such as water for injection, saline in physiological concentrations, or similar.

The present invention also comprises administration of a physiologically active conjugate comprising a TA1 peptide conjugated to a material which increases half-life of the TA1 peptide in serum of a patient when said conjugate is administered to a patient. The material may be a substantially non-antigenic polymer. Suitable polymers will have a molecular weight within a range of about 200-300,000, preferably within a range of about 1,000-100,000, more preferably within a range of about 5,000-35,000, and most preferably within a range of about 10,000-30,000, with a molecular weight of about 20,000 being particularly preferred.

The polymeric substances included are also preferably water-soluble at room temperature. A non-limiting list of such polymers include polyalkylene oxide homopolymers such as polyethylene glycol (PEG) or polypropylene glycols, polyoxyethylenated polyols, copolymers thereof and block copolymers thereof, provided that the water solubility of the block copolymers is maintained. Among the substantially non-antigenic polymers, mono-activated, alkyl-terminated polyalkylene oxides (PAO's), such as monomethyl-terminated polyethylene glycols (mPEG's) are contemplated. In addition to mPEG, C1-4 alkyl-terminated polymers may also be useful.

As an alternative to PAO-based polymers, effectively non-antigenic materials such as dextran, polyvinyl pyrrolidones, polyacrylamides, polyvinyl alcohols, carbohydrate-based polymers and the like can be used. Those of ordinary skill in the art will realize that the foregoing list is merely illustrative and that all polymer materials having the qualities described herein are contemplated. For purposes of the present invention, “effectively non-antigenic” means all materials understood in the art as being nontoxic and not eliciting an appreciable immunogenic response in mammals.

The polymer may be straight-chain or branched. Polyethylene glycol (PEG) is a particularly preferred polymer.

The polymer can be conjugated to the TA1 peptide by any suitable method. Exemplary methods for conjugating polymers to peptides are disclosed in U.S. Pat. Nos. 4,179,337, 4,766,106, 4,917,888, 5,122,614 and 6,177,074, as well as PCT International Publication No. WO 95/13090, all of which are incorporated herein by reference. Thymosin alpha 1 has five separate possible sites for amino group conjugation of a polymer, and polymer(s) can be conjugated at one or a plurality of sites. According to one embodiment, 20,000 molecular weight PEG is conjugated to the N-terminal end of TA1 to form a PEG-TA1. This can be formed by solid phase peptide synthesis of TA1 on insoluble polymeric support beads, as is known in the art, with appropriate side chain protective groups. After complete synthesis of the TA1 peptide on the beads, the protected TA1 is cleaved from the beads leaving the N-terminus with a free amino group, which is reacted with 20,000 molecular weight PEG. The side chain protective groups then are removed to form a conjugate in accordance with this embodiment of the invention.

The isolation, characterization and use of TA1 peptides is described, for example, in U.S. Pat. No. 4,079,127, U.S. Pat. No. 4,353,821, U.S. Pat. No. 4,148,788 and U.S. Pat. No. 4,116,951. Effective amounts of TA1 peptide can be determined by routine dose-titration experiments. TA1 has been found to be safe for humans when administered in doses as high as 16 mg/kg body weight/day. Preferred dosages of TA1 peptide are within the range of 0.001 mg/kg body weight/day to 10 mg/kg body weight/day. According to one embodiment, immune stimulating-effective amounts are at dosages which include the TA1 peptide in an amount within a range of about 0.1-20 mg. Preferred dosages include the TA1 peptide in an amount within the range of about 0.5-10 mg, more preferably about 1-5 mg, most preferably about 1.6-3.2 mg. The above dosages reflect only the TA1 peptide present in the composition, and not the weight of the polymer, if any, conjugated thereto.

Conjugation of a polymer to a TA1 peptide in accordance with the present invention substantially increases the plasma half-life of the peptide.

The TA1 peptide also can be administered with an effective amount of an interferon, such as interferon alpha, wherein interferon alpha-2b is preferred. Suitable dosages of interferon alpha-2b may be in the range of about 1-3 MU.

The TA1 peptide also can be administered with other immune stimulators or antiviral agents.

### Examples

### Example 1

### Effect of s.c. Treatment with Thymosin Alpha-1 on Inhibition of SARS-CoV Replication in Mice

Thymosin alpha-1 is a synthetic 28-amino acid peptide (N-acetyl-Ser-Asp-Ala-Ala-Val-Asp-Thr-Ser-Ser-Glu-Ile-Thr-Thr-Lys-Asp-Leu-Lys-Glu-Lys-Lys-Glu-Val-Val-Glu-Glu-Ala-Glu-Asn-OH (SEQ ID NO: 1)). Mice (n=10 per group) were treated with either 0.2 mg/kg or 2 mg/kg thymosin alpha-1 by subcutaneous (s.c.) injection or 50 μg/mouse of poly-ICLC (positive control) by the intranasal route (i.n.). Fifteen mice were treated s.c with thymosin alpha-1 buffer (saline) as a negative control. Thymosin alpha-1 was administered at the times and frequency indicated in Table 1. Poly-ICLC was administered 24 h before virus exposure and one time, 12 h, after virus exposure. Thymosin alpha-1 buffer (saline) was administered once at days one and two prior to virus exposure and at day one and two following virus exposure. No toxicity controls were included, because the doses used have previously been shown to be well tolerated.

For SARS-CoV virus infection, the mice were sedated with an i.p. injection of 100 mg/kg of ketamine® and mice were infected intranasally (i.n.) with 50 μl of clarified virus lysate diluted 1:5 in minimal essential medium. All treatments ceased two days after virus exposure, and animals were sacrificed at three days after virus infection. Lungs were removed and weighed. The lung was then homogenized and assayed for the presence of virus. Differences in mean lung weight and virus titers were analyzed by analysis of variance.

TABLE 1
Treatment regimens.

| | Mice/ | Compound | | | Treatment schedule |
|---|---|---|---|---|---|
| Group | group | Treatment | Drug Dosage | | (Relative to Virus Exposure) |
| 1 | 15 | thymosin alpha-1 buffer | 0.85% saline | | Day −2, −1, +1, +2 |
| 2 | 10 | thymosin alpha-1 | 2 | mg/kg | Day −2, −1, +1, +2 |
| 3 | 10 | thymosin alpha-1 | 0.2 | mg/kg | Same |
| 4 | 10 | thymosin alpha-1 | 2 | mg/kg | Day −2, −1 |
| 5 | 10 | thymosin alpha-1 | 0.2 | mg/kg | Same |
| 6 | 10 | thymosin alpha-1 | 2 | mg/kg | Day −2 |
| 7 | 10 | thymosin alpha-1 | 0.2 | mg/kg | Same |
| 8 | 10 | thymosin alpha-1 | 2 | mg/kg | −4, −3, −2, −1, |
| | | | | | 0*, +1, +2 (bid)* |
| 9 | 10 | thymosin alpha-1 | 0.2 | mg/kg | Same |
| 10 | 10 | Poly-ICLC | 50 | μg/mouse | −24 h, +12 h |

*Injections were 8 h apart.

All treatments with thymosin alpha-1 were very well tolerated, with all animals surviving and gaining weight (Table 2). Treatment with thymosin alpha-1 significantly reduced virus lung titers in mice infected with SARS-CoV when administered daily starting four days before virus exposure (Table 2). When mice were treated one time per day with thymosin alpha-1 at 2 mg/kg 48 and 24 h before virus exposure and subsequently once a day 24 and 48 h after virus exposure, virus lung titers were reduced by about one log10 unit. Dosing even more frequently (4, −3, −2, −1, 0*, +1, +2 (bid)) with thymosin alpha-1 at 2 and 0.2 mg/kg was also efficacious in reducing virus titers by approximately 0.5 log10 unit. The positive control, poly-ICLC, was effective in inhibiting the lung virus titers by nearly 3 log 10 units (P<0.01).

TABLE 2
Effects of s.c. Treatment with Thymosin Alpha-1
on the Replication of SARS-CoV (Urbani) in Mice

| | | | | Mean Body |
|---|---|---|---|---|
| Treatment | | | Virus Titer | Weight |
| Schedule | Dose | | (Log₁₀ CCID₅₀/g)^{a} | Change (g) |
| Day −2, −1, +1, +2 (qd)^{b} | 2 | mg/kg | 3.3 ± 0.6 | 1.6 |
| | 0.2 | mg/kg | 4.1 ± 0.6 | 1.4 |
| Day −2, −1 (qd) | 2 | mg/kg | 3.6 ± 0.4 | 1.4 |
| | 0.2 | mg/kg | 3.9 ± 0.7 | 1.9 |
| Day −2 (qd) | 2 | mg/kg | 4.1 ± 0.5 | 1.3 |
| | 0.2 | mg/kg | 3.8 ± 0.6 | 1.9 |
| −4, −3, −2, −1, | 2 | mg/kg | 3.5 ± 0.3* | 1.1 |
| 0^{c}, +1, +2 (BID) | | | | |
| | 0.2 | mg/kg | 3.7 ± 0.2* | 1.3 |
| Poly-ICLC | 50 | μg/mouse (i.n.) | 2.9 ± 0.1** | 0.0 |

| | | | |
|---|---|---|---|
| Day −2, −1, +1, +2 | thymosin alpha-1 | 4.4 ± 0.4 | 1.1 |
| | buffer | | |

^{a}Values are expressed as mean ± standard deviation.
^{b}The treatment is relative to virus exposure; “−” = day before virus exposure, “+” = day after virus exposure.
^{c}Compound was injected in mice immediately prior to virus exposure.
*P < 0.05,
**P < 0.01 compared to thymosin alpha-1 buffer control.

The results of this experiment show that thymosin alpha-1 is effective in reducing SARS-CoV lung titers in SARS infected mice, with virus titer reductions ranging from 0.5-1.0 log10 units.

1. A method for treating a respiratory coronavirus infection in a patient, comprising administering to said patient an effective amount of Thymosin alpha-1(TA1) sufficient to stimulate the patient's immune system, wherein the TA1 is administered at least once daily by subcutaneous injection.

2. The method of claim 1, wherein said respiratory viral infection is SARS.

3. The method of claim 1, wherein the effective amount of the TA1 is within the range of about 0.1 to 20 mg.

4. The method of claim 1, wherein the effective amount of the TA1 is within the range of about 0.5 to 10 mg.

5. The method of claim 1, wherein the effective amount of the TA1 is within the range of about 1 to 5 mg.

6. The method of claim 1, wherein the effective amount of the TA1 is 1.6 to 3.2 mg.

7. The method of claim 1, further comprising, administering an antiviral.

8. The method of claim 1, wherein the TA1 is conjugated to polyethylene glycol (PEG).

9. The method of claim 1, wherein the TA1 is synthetic or recombinant.

10. A method for stimulating the immune response in a patient at high risk of exposure to SARS, comprising administering to said patient an effective amount of Thymosin alpha-1 (TA1) sufficient to stimulate the patient's immune system, wherein the TA1 is administered at least once daily by subcutaneous injection.

11. The method of claim 10, wherein the effective amount of the TA1 is within the range of about 0.1 to 20 mg.

12. The method of claim 10, wherein the effective amount of the TA1 is within the range of about 0.5 to 10 mg.

13. The method of claim 10, wherein the effective amount of the TA1 is within the range of about 1 to 5 mg.

14. The method of claim 10, wherein the effective amount of the TA1 is 1.6 to 3.2 mg.

15. The method of claim 10, further comprising, administering an antiviral.

16. The method of claim 10, wherein the TA1 is conjugated to polyethylene glycol (PEG).

17. The method of claim 10, wherein the TA1 is synthetic or recombinant.
